(19) United States
(12) Patent Application Publication
(10) Pub. No.: US 2013/0274247 A1
(43) Pub. Date: Oct. 17, 2013

(54) Mirtazapine as an Appetite Stimulant for Cats

(71) Applicants: Jessica Quimby, Fort Collins, CO (US); Katharine Lunn, Raleigh, NC (US)

(72) Inventors: Jessica Quimby, Fort Collins, CO (US); Katharine Lunn, Raleigh, NC (US)

(21) Appl. No.: 13/486,278

(22) Filed: Jun. 1, 2012

Related U.S. Application Data
(60) Provisional application No. 61/625,421, filed on Apr. 17, 2012.

(51) Int. Cl. A61K 31/55; A61P 1/14

(52) U.S. Cl. 514/214.02

(57) ABSTRACT

Methods for stimulating appetite in cats in need thereof are disclosed.

This application claims priority to U.S. Provisional Application No. 61/625,421 filed Apr. 17, 2012, the entire disclosure being incorporated herein by reference as though set forth in full.

### FIELD OF THE INVENTION

This invention relates to the fields of pharmacology and feline health. More specifically, the invention provides compositions and methods effective to enhance appetite and nutrition in cats in need thereof.

### BACKGROUND OF THE INVENTION

Several publications and patent documents are cited throughout the specification in order to describe the state of the art to which this invention pertains. Each of these citations is incorporated herein by reference as though set forth in full.

Chronic kidney disease (CKD) is common in geriatric cats. The kidneys are responsible for excretion of gastrin and as renal function deteriorates, gastrin concentrations may increase, leading to uremic gastritis (1). Other factors may contribute to lethargy and inappetence in these patients, including metabolic acidosis, anemia, and renal secondary hyperparathyroidism (2). As a result of these factors, cats with CKD frequently experience anorexia and vomiting. Inappetence can lead to negative energy balance with associated weight loss, muscle weakness, and poor quality of life. In addition, recent studies have documented the therapeutic value of specially formulated diets in the management of CKD (3-5). These diets typically contain restricted amounts of high quality protein, adequate non-protein calories, and are restricted in phosphorus (3). Failure of the patient to eat negates the benefit of dietary management, and therefore a key therapeutic target for these patients is the maintenance of appetite and food intake. Current strategies to enhance appetite include the use of H2-receptor antagonists or protonpump inhibitors to manage uremic gastritis, and cyproheptadine as an appetite stimulant (2). Feeding tubes also may be used, but are not an acceptable option for many pet owners.

Clearly, a need exists in the art for agents that stimulate appetite in diseased or geriatric cats.

### SUMMARY OF THE INVENTION

In accordance with the present invention, a method for the enhancing appetite in cats in need of such treatment is disclosed. An exemplary method entails administering a therapeutically effective amount of mirtazapine or a pharmaceutically acceptable salt thereof to a cat, said mirtazapine being administered at a dosage between 1.0 and 2.5 mg. In a preferred embodiment, 1.88 mg is administered.

The methods of the invention are particularly useful for stimulating appetite in cats having chronic kidney disease. In this embodiment, the mirtazapine is administered every other day. Geriatric cats can also benefit using the methods of the invention. Mirtazapine can be administered via many routes including without limitation, systemically, parenterally, orally, or via subcutaneous injection. In a preferred aspect of the method of the invention, mirtazapine is administered orally.

### BRIEF DESCRIPTION OF THE DRAWINGS

FIG. 1. Effect of mirtazapine on weight. A statistically significant increase in weight was seen in CKD cats administered 1.88 mg mirtazapine every other day for three weeks (p=0.002).

FIG. 2. Effect of mirtazapine on appetite. A statistically significant increase in appetite score was seen in CKD cats administered 1.88 mg mirtazapine every other day for three weeks (p=0.02).

FIG. 3. Effect of mirtazapine on activity. A statistically significant increase in activity was seen in CKD cats administered 1.88 mg mirtazapine every other day for three weeks (p=0.02).

FIG. 4. Effect of mirtazapine on vomiting. A statistically significant decrease in vomiting was seen in CKD cats administered 1.88 mg mirtazapine every other day for three weeks (p=0.047).

FIG. 5. Drug concentration curves for mirtazapine in CKD cats (n=6) and age-matched controls (AMC) cats (n=6). Age appears to affect the metabolism of the drug, but does not entirely explain the increased exposure in cats with CKD.

### DETAILED DESCRIPTION OF THE INVENTION

Mirtazapine, an antidepressant used in humans, has gained popularity in veterinary medicine because of its anti-emetic and appetite-stimulating properties (6, 7). These effects appear to be a result of antagonism of the 5-HT3 receptor, which is important in the physiology of emesis (8). A recent placebo-controlled crossover study in young normal cats demonstrated that mirtazapine is an effective appetite stimulant in this species (9). The efficacy of commonly administered doses was examined and although in both 1.88 mg/cat and 3.75 mg/cat resulted in increased food consumption as compared to placebo, more undesirable effects (increased vocalization, activity, and socialization) were seen at the higher dose. Other reported adverse effects in cats are mild and dose dependent, and include hyperexcitability and muscle tremors (10). Previously, mirtazapine doses for cats and dogs were extrapolated from human medicine and adjusted based on clinical observations. A recent study in young normal cats indicated that the half-life of the drug was shorter than that previously suspected (9). In addition, the drug does not appear to display linear pharmacokinetics in cats, and larger doses may result in a longer half-life. Pharmacokinetic data from humans have demonstrated that a number of factors affect the metabolism of mirtazapine, including sex and age, and hepatic or renal impairment (8). The latter is likely because the drug undergoes hepatic metabolism and renal excretion.

In accordance with the present invention, we have determined the pharmacokinetics of mirtazapine in cats with CKD and in age-matched controls (AMC) to investigate the effects of renal impairment in this species and mirtazapine treatment in this patient cohort.

### DEFINITIONS

The phrase “chronic kidney disease (CKD) refers to chronic renal failure and is one of the most common conditions affecting older cats. In most cases, chronic renal failure is progressive over time so that there is a gradual advancement and worsening of the disease. The rate of progression of the disease varies considerably between individuals. Appropriate support and treatment can both increase the quality of life of affected cats and prolong life by slowing down the progression of the disease.

As used herein, the term “pharmaceutically effective” refers to that amount of mirtazapine, which enhances the appetite in anorexic cats, particularly in elderly and cats with chronic kidney disease. For example, a pharmaceutically effective amount for enhancing the appetite in cats in need of such treatment refers to the amount which when administered increases appetite relative to cats not receiving the drug. The precise therapeutic dosage of mirtazapine necessary to be pharmaceutically active will vary with age, size, sex and condition of the subject, the nature and severity of the disorder or disease to be treated. The appropriate amounts have been determined for stimulating the appetite in normal cats and cats having CKD. The present inventors have determined that smaller more frequent doses (˜2 mg/day) are suitable for administration to normal feline patients. In contrast, the present inventors have determined that CKD appears to slow the clearance of mirtazapine, accordingly every other day dosing is recommended for cats suffering from this disorder.

The following materials and methods are provided to facilitate the practice of the present invention.

### Animals

In this prospective pharmacokinetic study, 6 client-owned stable CKD cats, 2 each from International Renal Interest Society (IRIS) stages II, III and IV, 11 and 6 age-matched (within 6 months) healthy geriatric control cats (AMC) were enrolled by stratified convenience sampling. There were 4 spayed females and 2 neutered males in the CKD group and 3 spayed females and 3 neutered males in the AMC group. Cats were considered to have stable CKD if serum creatinine concentration had not changed by more than 10% on at least 2 measurements in the previous 60 days. Diagnostic tests required before enrollment included a minimum database consisting of serum biochemistry profile, CBC, urinalysis, urine culture, blood pressure, and serum total thyroxine concentration. Healthy control cats were defined as those with no clinical abnormalities, normal laboratory test results including serum creatinine <1.8 mg/dL and urine specific gravity >1.035. Exclusion criteria included other systemic illnesses, complications of CKD such as hypertension, pyelonephritis or ureteral obstruction, or decompensation of CKD requiring hospitalization and IV fluid therapy. The project was approved by the Institutional Animal Care and Use Committee at Colorado State University, and all owners gave written informed consent before participation.

### Drug Preparation

Commercially available generic 15 mg mirtazapine^{a} tablets were compounded into 1.88 mg doses by the pharmacy at the Colorado State University Veterinary Medical Center according to the Professional Compounding Centers of America® protocol as previously described (9). The method used is guaranteed to produce accurate compounding to within 10% of the target dose. Analysis of random compounded capsules for mirtazapine content using liquid chromatography coupled to tandem mass spectrometry (LC/MS/MS) showed accuracies of 94.5±4.6% to the intended content and stability of at least 6 months as formulated. Mirtazapine capsules were compounded within 1 month of use and stored at room temperature.

### Sample Collection

The cats were fasted for 12 hours before beginning the study. A jugular catheter was placed under ketamineb (20 mg per cat IV) and butorphanolb (0.1 mg/kg IV) sedation 3 hours before mirtazapine administration, to allow for ease of sample collection. A capsule containing 1.88 mg of mirtazapine was administered PO once, followed by 3 mL of water administered by syringe. Blood samples (1.0 mL) were obtained before, and 0.5, 1, 1.5, 2, 4, 8, 24, and 48 hours after mirtazapine administration. Samples were centrifuged within 10 minutes of collection and serum was harvested and stored at _{—}80° C. until analysis.

### Mirtazapine Analysis

Mirtazapine was measured using LC/MS/MS. Analysis was carried out in the Pharmacology Core at the Colorado State University Veterinary Medical Center using a previously developed and validated LC/MS/MS-based assay for the analysis of mirtazapine in cat serum (9). Assay performance for each batch was assessed utilizing at least 10% quality assurance, quality control (QA/QC) samples dispersed among unknown samples at low (1 ng/mL), mid (10 ng/mL) and high (100 ng/mL) ranges of the standard curve (0.5-500 ng/mL) with batches failing if >25% of the QA/QC samples were outside of the accepted level of 85% accuracy. Accuracy of QA/QC samples among the batches analyzed for this study ranged from 94.5±4.6% to 92.2±6.8%. The lower limit of quantitation (LLOQ) for this assay was based on the level of detection with >85% accuracy and a coefficient of variation (%)<15%, and was determined to be 0.5 ng/mL. Assay performance was linear to >500 ng/mL, but 500 ng/mL was used as the upper limit of the assay as utilized because of a lack of samples exceeding this concentration.

### Pharmacokinetic Analysis

Pharmacokinetic analysis was performed using a non-compartmental method. Area under the curve to infinity (AUC_{∞}), disappearance half-life (t½^{λ}), time to maximum serum concentration (Tₘₐₓ), and maximum serum concentration (Cₘₐₓ) were calculated. Because mirtazapine was administered by an extravascular route, absorbed dose is equal to D (dose)×bioavailability (F). Thus, parameters in which the calculation is based on the assumption that 100% of the dose reaches the systemic circulation (clearance [CL] and volume of distribution [Vd]) are expressed as CL corrected for bioavailability or clearance/bioavailability (CL/F) and volume of distribution/bioavailability (Vd/F).12 Using the term CL/F and comparing it between the 2 treatment groups assumes that F, or bioavailability, is not different between the 2 treatment groups. The accumulation factor at steady state after multiple doses was estimated from the pharmacokinetic data using the equation: Accumulation Factor=1/(1−e−Kel*T). The terminal elimination rate was used for estimating the accumulation factor as the applicable kel, and the dosing interval (T) was set at 24 or 48 hours.

### Statistical Analysis

Comparison of pharmacokinetic parameters between the CKD and AMC cat groups was performed using a Mann-Whitney U-test. Prism software© was used for all analyses.

The following example is provided to illustrate particular embodiments of the invention. It is not intended to limit the invention in any way.

### Example I

Descriptive statistics for the 12 cats enrolled, including age, dosage (mg/kg), and serum creatinine concentration, are presented in Table 1. Two cats in IRIS Stage II (serum creatinine concentrations 2.4 and 2.5 mg/dL), 2 cats in IRIS Stage III (serum creatinine concentrations 2.9 and 3.3 mg/dL), and 2 cats in IRIS Stage IV (serum creatinine concentrations 5.7 and 6.1 mg/dL) were enrolled in the CKD group. Statistically significant differences were not detected in age or mg/kg dosage between the 2 groups. There was a statistically significant difference in serum creatinine concentration between the 2 groups (P=0.002).

### Pharmacokinetic Analysis

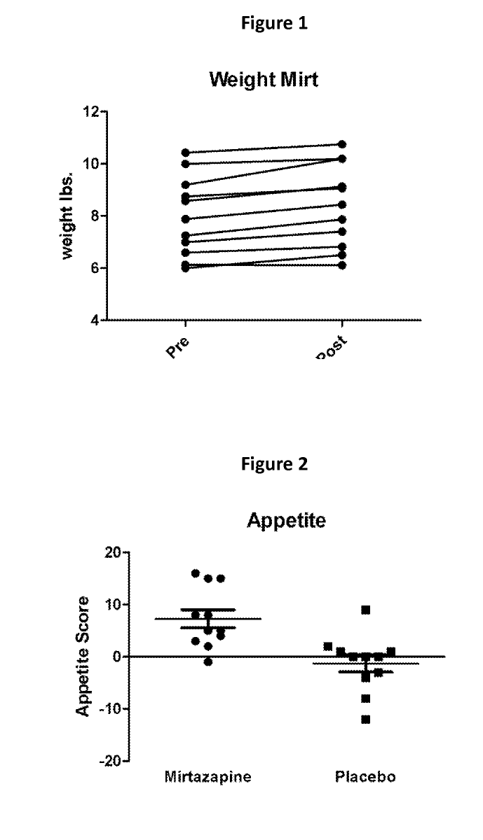

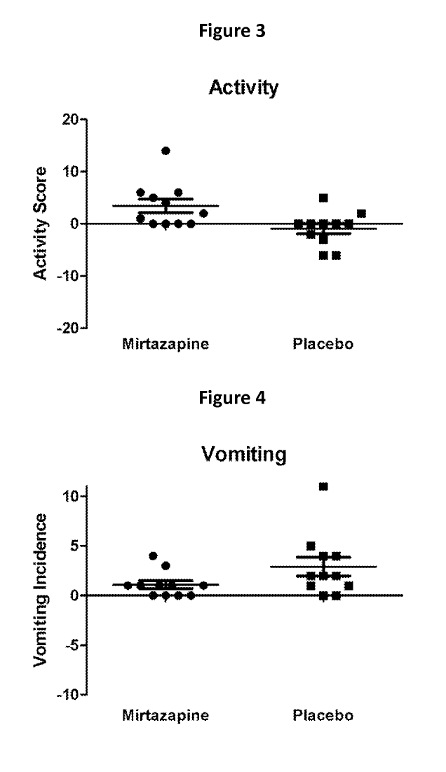

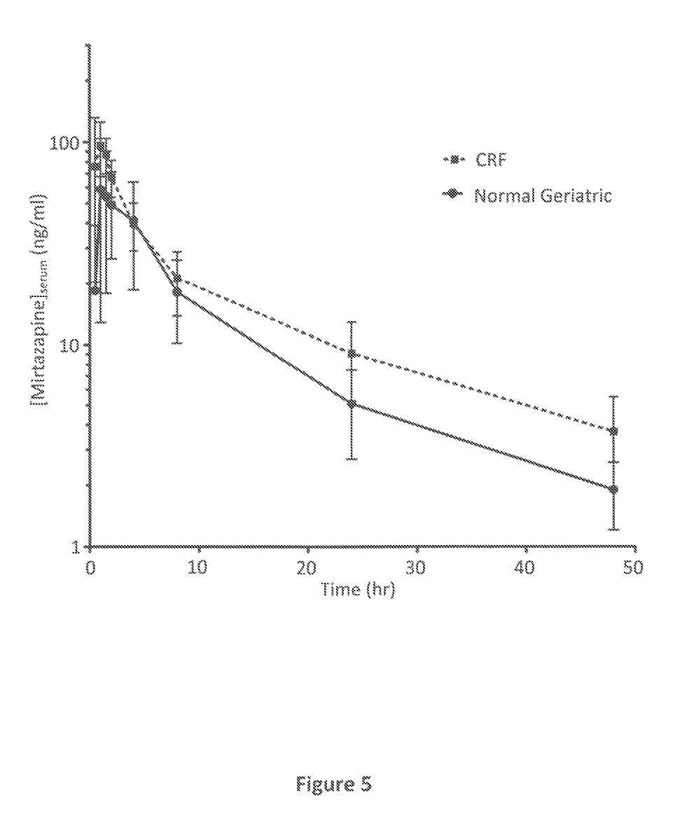

Pharmacokinetic parameters are shown in Table 2. There was a statistically significant difference in AUC_{∞} and CL/F between the AMC cats and CKD cats. Graphical representations of drug concentration curves for AMC cats and CKD cats are illustrated in FIGS. 1-5.

TABLE 1
Descriptive Statistics of Enrolled Healthy Geriatric
Cats and Cats with Chronic Kidney Disease

| Pharmacokinetic | Healthy Geriatric | | | Chronic kidney disease | | |
|---|---|---|---|---|---|---|
| Parameter | Median | Range | Mean ± SD | Median | Range | Mean ± SD |
| Age | 10.7 | 7.8-13.8 | 10.8 ± 2.3 | 11 | 8.3-13.7 | 11 ± 2.2 |
| Mg/kg dose | 0.43 | 0.33-0.58 | 0.44 ± 0.08 | 0.45 | 0.4-0.78 | 0.51 ± 0.15 |
| Creatinine | 1.4 | 0.7-1.8 | 1.3 ± 0.4 | 3.1 | 2.4-6.1 | 3.8 ± 1.6 |

TABLE 2
Pharmacokinetic Parameters of Orally-Dosed Mirtazapine in
Healthy Geriatric Cats and Cats with Chronic Kidney Disease

| Pharmacokinetic | Healthy Geriatric | | | Chronic kidney disease | | |
|---|---|---|---|---|---|---|
| Parameter | Median | Range | Mean ± SD | Median | Range | Mean ± SD |
| Cₘₐₓ (ng/mL) | 83.6 | 50.2-103 | 79.6 ± 21.7 | 109.5 | 79.1-164 | 110.6 ± 30.8 |
| Cₘₐₓ/Dose (ng/mL)/ | 179.1 | 132.3-210.1 | 180.3 ± 44.3 | 221.6 | 154.4-278.3 | 219.6 ± 44.7 |
| (mg/kg) | | | | | | |
| Tₘₐₓ (hr) | 1 | 1-4 | 2 ± 1.5 | 1 | 0.5-1.5 | 1 ± 0.3 |
| Half life (hr) | 12.0 | 10.1-15.4 | 12.3 ± 1.8 | 15.8 | 10.8-24.8 | 16.4 ± 5.1 |
| AUC_{∞} * (ng/mL · hr) | 560.8 | 400.4-941.2 | 589.8 ± 185.3 | 828.4 | 597.1-1253.6 | 866.5 ± 257.9 |
| AUC_{∞}/Dose * | 1375.6 | 926.5-1612.2 | 1320.4 ± 236.0 | 1676.8 | 1355.1-2229.3 | 1701.2 ± 301.3 |
| (ng/mL · hr)/(mg/kg) | | | | | | |
| CL/F * (L/hr/kg) | 0.73 | 0.62-1.1 | 0.79 ± 0.16 | 0.6 | 0.45-0.74 | 0.61 ± 0.1 |
| Vd [symbol] /F (L) | 14.4 | 10.1-15.3 | 13.9 ± 3.2 | 13.4 | 11.5-16.1 | 13.6 ± 2.0 |

[symbol]

indicates data missing or illegible when filed

### Assessment of Accumulation

Drug accumulation was calculated for 24- and 48-hour dosing intervals for both CKD and AMC groups. For the CKD cats, an accumulation factor of 1.57 was calculated for 24-hour dosing and an accumulation factor of 1.15 was calculated for 48-hour dosing. For the AMC cats, an accumulation factor of 1.35 was calculated for 24-hour dosing and an accumulation factor of 1.07 was calculated for 48-hour dosing.

### Discussion

In the present study, the pharmacokinetics of mirtazapine in CKD cats and AMC were explored. A significant difference in drug exposure (AUC) and CL/F of mirtazapine was found between CKD cats and AMC cats. Mirtazapine is a 5-HT3 receptor antagonist with appetite-stimulating properties. We have previously demonstrated that a dose of 1.88 mg significantly stimulates appetite in young normal cats (9). This dose was associated with a half-life of approximately 10 hours, thus allowing daily dosing with little drug accumulation (9). In comparison to our previously reported mean half-life in normal cats (10 hours), the mean half-life of mirtazapine in this study was approximately 12 hours for AMC cats and 15 hours for CKD cats. The mean AUC in normal cats previously was reported to be 397 ng/mL/hr, in comparison to 523.9 ng/mL/hr in AMC cats and 686.5 ng/mL/hr in CKD cats in the present study. Although there was no significant difference in mirtazapine dose between the CKD and AMC cats, the statistical power of the comparison between doses in each group is limited attributable to sample size. Therefore, AUC and Cₘₐₓ also were calculated with dose adjustment (Table 2) to decrease the possible effect of difference in dose between the 2 groups. Even with this adjustment, AUC still was significantly different between the AMC and CKD groups. The mean CL/F previously was found to be 1.1 L/hr/kg in young healthy cats (9), in comparison to 0.79 L/hr/kg in AMC cats and 0.61 L/hr/kg in CKD cats in the present study. From this information, we suggest that although age appears to have some influence on the metabolism of the drug, it cannot entirely account for the difference between CKD and young normal cats. Therefore, we conclude that CKD delays the CL/F of mirtazapine in cats.

In the human medical literature, moderate to severe renal disease is reported to increase mirtazapine exposure (AUC) caused by a decrease in drug CL.13 A similar relation may exist between renal disease and mirtazapine CL/F in cats, based on the data presented herein and that in a previous study (9). When the data from the 2 studies were combined, there was a significant negative correlation between serum creatinine concentration and CL of mirtazapine (r=_{—}0.69 with P=0.0024) when data from young normal cats (9), normal geriatric cats, and CKD cats were analyzed using Spearman rank correlation. Elimination half-life of mirtazapine is unaffected by the severity of renal disease in humans. In this study, although a significant difference in half-life was not detected between AMC and CKD cats, perhaps attributable to small sample size, there is a difference between these data and that reported in normal cats (9). It is unknown to what extent nonlinear pharmacokinetics may play a role in this observation, because half-life would be expected to be prolonged with increased exposure. As in humans (8), differences in metabolism attributable to age also may play a role. In this data set, pharmacokinetic parameters for the AMC group, particularly AUC, vary notably from those reported for young normal cats (9).

Two cats from each IRIS stage were included in the present study to represent the range of renal function encountered in clinical practice. This likely contributed to greater standard deviation (SD) for some parameters, including half-life, and potentially affected our ability to find a significant difference between groups. If cats from only 1 IRIS stage had been studied, variability may have been decreased, and it may have been possible to demonstrate a difference between half-life in AMC and CKD cats.

In humans, sex is known to affect mirtazapine pharmacokinetics, with shorter half-life and lower AUC in young men compared to women (8). Thus, an effort was made to control for this factor by having approximately equal numbers of both sexes in each group. Unfortunately, complete age and sex matching was not possible because of difficulty in recruiting CKD patients without concurrent illness, and this potentially could have affected results. Study participant numbers were too small to determine if there was a significant effect of sex on metabolism of mirtazapine in cats.

The pharmacokinetic information obtained in this study can be used to help determine dose intervals for cats with CKD. Calculation of an accumulation factor for daily dosing compared to every other day dosing was performed. Although no evidence of drug accumulation was seen with the 48-hour dose interval (accumulation factor=1.15), accumulation potentially is possible with daily dosing in CKD cats (accumulation factor=1.57). This is in contrast to young normal cats, where no evidence of drug accumulation was found with daily dosing (accumulation factor=1.2) (9). However, because concentration may not reflect clinical effect, to fully understand the pharmacokinetic and pharmacodynamic implications of this dosing regimen in CKD cats, a clinical trial with repeated dosing every 48 hours should be performed.

In conclusion, chronic kidney disease is common in elderly cats and these patients typically suffer from poor appetite. Enhancing their appetite and keeping nutrition adequate is an important part of long-term patient management. Mirtazapine is a novel appetite-stimulant and anti-emetic drug that we demonstrate increases appetite in cats, particularly in cats with CKD. We also show that CKD in cats results in higher drug exposure and appears to slow the CL/F of mirtazapine. This information should be considered when clinicians are determining dosing regimens for their patients.

### REFERENCES

1. Goldstein R E, Marks S L, Kass P H, et al. Gastrin concentrations in plasma of cats with chronic renal failure. J Am Vet Med Assoc 1998; 213:826-828.
2. Plotnick A. Feline chronic renal failure: Long-term medical management. Compend Contin Educ Vet 2007; 29:342-344.
3. Elliott J, Rawlings J M, Markwell P J, et al. Survival of cats with naturally occurring chronic renal failure: Effect of dietary management. J Small Anim Pract 2000; 41:235-242.
4. Ross S J, Osborne C A, Kirk C A, et al. Clinical evaluation of dietary modification for treatment of spontaneous chronic kidney disease in cats. J Am Vet Med Assoc 2006; 229:949-957.
5. Planting a E A, Everts H, Kastelein A M, et al. Retrospective study of the survival of cats with acquired chronic renal insufficiency offered different commercial diets. Vet Rec 2005; 157:185-187.
6. Kast R E, Foley K F. Cancer chemotherapy and cachexia: Mirtazapine and olanzapine are 5-HT3 antagonists with good antinausea effects. Eur J Cancer Care Engl) 2007; 16:351-354.
7. Pae C U. Low-dose mirtazapine may be successful treatment option for severe nausea and vomiting. Prog Neuropsychopharmacol Biol Psychiatry 2006; 30:1143-1145.
8. Timmer C J, Sitsen J M, Delbressine L P. Clinical pharmacokinetics of mirtazapine. Clin Pharmacokinet 2000; 38:461-474.
9. Quimby J M, Gustafson D L, Samber B J, et al. Studies on the pharmacokinetics and pharmacodynamics of mirtazapine in healthy young cats. J Vet Pharmacol Ther 2011; 34:388-396. doi: 101111/j1365-2885201001244x.
10. Cahill C. Mirtazapine as an antiemetic. Veterinary Forum 2006:34-36.
11. Polzin D J, Osborne C A, Ross S J. Chronic kidney disease. In: Ettinger S J, Feldman E C, eds. Textbook of Veterinary Internal Medicine, 6th ed. St Louis, Mo.: WB Saunders; 2005:1756-1785.
12. Wagner J. Pharmacokinetics for the Pharmaceutical Scientist. Lancaster, Pa.: Technomic Publishing Company, Inc; 1993.
13. Bengtsson F, Hoglund P, Timmer C J, et al. Mirtazapine oral single dose kinetics in patients with different degrees of renal failure. Human Psychopharmacolog 1998; 13:357-365.
14. Baxmann A C, Ahmed M S, Marques N C, et al. Influence of muscle mass and physical activity on serum and urinary creatinine and serum cystatin C. Clin J Am Soc Nephrol 2008; 3:348-354.

While certain preferred embodiments of the present invention have been described and specifically exemplified above, it is not intended that the invention be limited to such embodiments. Various modifications may be made to the invention without departing from the scope and spirit thereof as set forth in the following claims.

1. A method for enhancing appetite in a cat having chronic kidney disease, comprising administering mirtazapine or a pharmaceutically acceptable salt thereof to said cat, in an amount between 1.0 and 2.5 mg every other day.

2. The method of claim 1, wherein 1.88 mg is administered.

3. (canceled)

4. The method of claim 1, wherein said cat is geriatric.

5. The method of claim 1, wherein mirtazapine is administered orally to a cat.

6. The method of claim 1, wherein said cat is anorexic.

7. A method for enhancing appetite in a cat in need of such treatment, comprising administering mirtazapine or a pharmaceutically acceptable salt thereof to said cat at a dosage of approximately 1.88 mg.

8. The method of claim 7, wherein said cat has chronic kidney disease, and said mirtazapine is administered every other day.

9. The method of claim 7, wherein said cat is anorexic.
